(19) United States
(12) Patent Application Publication
(10) Pub. No.: US 2024/0180815 A1
(43) Pub. Date: Jun. 6, 2024

(54) METHODS FOR RESISTING SKIN AGING BY USING KIWIBERRY EXTRACT

(71) Applicant: TCI CO., LTD., Taipei (TW)

(72) Inventors: YUNG-HSIANG LIN, Taipei (TW); HSIAO-NAI LIN, Taipei (TW)

(21) Appl. No.: 18/528,783

(22) Filed: Dec. 5, 2023

Related U.S. Application Data
(60) Provisional application No. 63/386,175, filed on Dec. 6, 2022.

(30) Foreign Application Priority Data
Nov. 27, 2023 (TW) 112145903

(51) Int. Cl. A61K 8/9789; A61Q 19/08

(52) U.S. Cl. CPC A61K 8/9789; A61Q 19/08

(57) ABSTRACT

A method for resisting skin aging is provided. The method includes administering to a subject in need thereof a composition that includes an effective dose of kiwiberry extract. The kiwiberry extract is obtained by extracting kiwiberry (Actinidia arguta) with water. The kiwiberry extract achieves the effect of resisting skin aging by reducing skin damage, reducing damage to DNA structure, or a combination thereof.

### CROSS-REFERENCES TO RELATED APPLICATIONS

This application claims the benefit of U.S. provisional application Ser. No. 63/386,175, filed on Dec. 6, 2022 and claims the priority to Patent Application No. 112145903 filed in Taiwan, R.O.C. on Nov. 27, 2023. The entirety of the above-mentioned patent applications are hereby incorporated by references herein and made a part of the specification.

### REFERENCE OF AN ELECTRONIC SEQENCE LISTING

The contents of the electronic sequence listing (P223934USI.xml; Size: 4,716 bytes; and Date of Creation: Nov. 30, 2023) is herein incorporated by reference in its entirety.

### BACKGROUND

### Technical Field

The present invention relates to a kiwiberry extract, and in particular to a kiwiberry extract with a function of resisting skin aging.

### Related Art

With the changes of the times, people pursue a perfect appearance, with very high requirements for appearance outline, skin quality, skin elasticity, skin texture and condition externally, and collagen content internally. Healthy skin and its complexion are a major factor in maintaining a radiant appearance. Therefore, people are increasingly paying attention to skin health care and conditioning to maintain the best condition from inside out.

In order to solve the above problems, those skilled in the art urgently need to develop a functional food that can solve the above problems to benefit a vast number of people in need thereof.

### SUMMARY

In view of this, the present invention provides a kiwiberry extract made from kiwiberry (Actinidia arguta), which has a function of resisting skin aging.

In some embodiments, provided is a method for resisting skin aging, including administering to a subject in need thereof a composition that includes an effective dose of kiwiberry extract. The kiwiberry extract is obtained by extracting kiwiberry (Actinidia arguta) with water.

In some embodiments, provided is a use of a kiwiberry extract for preparing a composition for resisting skin aging. The kiwiberry extract is obtained by extracting kiwiberry (Actinidia arguta) with water.

In some embodiments, the kiwiberry extract is obtained by extracting kiwiberry in water at 70° C. to 90° C. for 90 min to 120 min.

In some embodiments, the kiwiberry extract is obtained by extracting kiwiberry in water at 70° C. to 90° C. until a Brix degree thereof reaches 20° C. is 0.6±0.5 at 20° C.

In some embodiments, the weight ratio of the kiwiberry to the water is (1 to 5):(5 to 10).

In some embodiments, the kiwiberry extract reduces skin damage, reduces damage to DNA structure, or a combination thereof to achieve the effect of resisting skin aging.

In some embodiments, the kiwiberry extract achieves reduction of skin damage by reducing reactive oxygen species (ROS).

In some embodiments, the kiwiberry extract achieves reduction of damage to DNA structure by increasing expression of an NAD-dependent deacetylase (Sirtuin-1, SIRT1) gene and/or an 8-hydroxyguanine glycosylase (OGG1) gene in the subject.

In some embodiments, the kiwiberry extract reduces skin laxity to achieve the effect of resisting skin aging.

In some embodiments, the kiwiberry extract improves skin elasticity to achieve the effect of resisting skin aging.

In some embodiments, the kiwiberry extract soothes skin redness to achieve the effect of resisting skin aging.

In some embodiments, when the composition is in liquid form, the effective dose of the composition is 2 g/day, or when the composition is in solid form, the effective dose of the composition is 0.2 g/day.

In summary, the kiwiberry extract of the embodiments of the present disclosure is prepared by extracting the kiwiberry with the water, and can be used for preparing the composition for resisting skin aging. In some embodiments, the kiwiberry extract reduces skin damage by reducing reactive oxygen species (ROS) in the subject, and reduces damage to DNA structure by increasing expression of SIRT1 gene and/or OGG1 gene in the subject, or a combination thereof. In some embodiments, the kiwiberry extrac reduces skin laxity of the subject, improves skin elasticity of the subject, soothes skin redness of the subject, or a combination thereof.

### BRIEF DESCRIPTION OF THE DRAWINGS

FIG. 1 is a bar chart showing the relative expression levels of SIRT1 gene;

FIG. 2 is a bar chart showing the relative content of reactive oxygen species (ROS);

FIG. 3 is a bar chart showing the relative level of damage to cell DNA structure;

FIG. 4 is a bar chart showing the relative expression levels of SIRT1 gene of the subjects at week 0 and week 4;

FIG. 5 is a bar chart showing the relative expression levels of OGG1 gene of the subjects at week 0 and week 4;

FIG. 6 is a bar chart showing the relative degrees of skin laxity of the subjects at week 0 and week 4;

FIG. 7 is a bar chart showing the relative degrees of skin elasticity of the subjects at week 0 and week 4;

FIG. 8 is a bar chart showing the relative degrees of skin redness of the subjects at week 0 and week 4; and

FIG. 9 is photographs showing the conditions of skin redness of one of the subjects at week 0 and week 4.

### DETAILED DESCRIPTION

Excel software is used for statistical analysis herein. Data is represented by mean±standard deviation (SD), and differences between groups are analyzed with student's t-test. In the figures, “*” or “#” represents that p value is less than 0.05, “**” or “##” represents that p value is less than 0.01, and “***” or “###” represents that p value is less than 0.001. The more the “*” or “#”, the more significant the statistical differences.

The term “extract” refers to a product prepared by extraction. The extract may be presented in the form of a solution dissolved in a solvent, or it may be presented as a concentrate or essence without or substantially without a solvent, or it may be presented as dried powder.

Kiwiberry (scientific name: Actinidia arguta var. cordifolia; English: Kiwiberry) is a hardy kiwi, also known as mini kiwifruit, which is a plant of Genus Actinidiae in Family Actinidiaceae, and is one of the most successful wild fruit tree varieties domesticated and cultivated from 1901 to 2000. The kiwiberry is a close relative of kiwifruits, with a volume 3 to 4 times smaller than the kiwifruit, but its vitamin C content is 1.3 times that of the kiwifruit. Furthermore, the kiwiberry also contain polyphenolic compounds, carotenoid, trace elements, and other substances.

In some embodiments, after fruits of kiwiberry (Actinidia arguta) are roughly crushed, the crushed fruits are mixed with an extraction solvent (e.g., water) in a certain weight ratio, and then, the mixture is extracted at a specific temperature to obtain a mixed solution containing solids. Subsequently, an initial extraction solution is filtered to remove solid trace impurities, and subsequently, the filtered mixed solution is concentrated to obtain a concentrated solution. Subsequently, the concentrated solution is sterilized to obtain a sterilized liquid kiwiberry extract. The concentrated solution is dried into powder by spray drying to obtain a solid kiwiberry extract. For example, the extraction solvent may be water; the certain weight ratio may be (1 to 5):(5 to 10); the specific temperature may be 70° C. to 90° C.; and specific time may be 90 min to 120 min.

In some embodiments, the fruit of the kiwiberry includes peel, flesh, and seeds.

Here, a specific proportion of extraction solvent and a substance to be extracted (such as the crushed fruits) or specific extraction time can significantly improve the extraction efficiency; and the specific extraction time can avoid the possible degradation of active ingredients in the extract due to overlong extraction time.

In some embodiments, the kiwiberry extract is prepared by roughly crushing fruits of kiwiberry and then mixing the crushed fruits with water in a weight ratio of (1 to 5):(5 to 10), and extracting the mixture at 70° C. to 90° C. for 90 min to 120 min. For example, the kiwiberry extract is prepared by mixing fruit powder of kiwiberry roughly crushed to 12 mm, with water in a weight ratio of 1:10 and extracting the mixture at 85° C.±5° C. for about 90 min.

In some embodiments, the kiwiberry extract is obtained by extracting kiwiberry in water at 70° C. to 90° C. until a mixed solution meets an established specification, and the established specification is that Brix of the mixed solution at 20° C. is 0.6±0.5. The weight ratio of the kiwiberry to the water is (1 to 5):(5 to 10). For example, fruit powder of the kiwiberry roughly crushed to 12 mm is mixed with the water in a weight ratio of 1:10, and the mixture is extracted at 85° C.±5° C. for about 90 min to obtain a mixed solution, and the Brix of the mixed solution is measured to confirm whether it meets an established specification or not. The aforementioned established specification is that the Brix of the mixed solution at 20° C. is 0.6±0.5.

In some embodiments, the mixed solution is filtered through a 400-mesh filter to remove fine solids, and the filtered mixed solution is subjected to vacuum concentration to obtain the kiwiberry extract. For example, the temperature of vacuum concentration may be 60° C.±5° C.

In some embodiments, vacuum concentration is performed by using vapor of 1±0.2 kg/cm².

In some embodiments, the kiwiberry extract has a function of resisting skin aging. For example, the resisting skin aging is to reduce skin damage, reduce damage to DNA structure, or a combination thereof. Specifically, the kiwiberry extract reduces the skin damage by reducing reactive oxygen species (ROS). The kiwiberry extract reduces the damage to DNA structure by increasing expression of a NAD-dependent deacetylase sirtuin-1 (SIRT1) gene and/or an 8-hydroxyguanine glycosylase (OGG1) gene.

In some embodiments, the kiwiberry extract can effectively enhance an anti-aging gene, and the anti-aging gene may be the SIRT1 gene. Protein encoded by the SIRT1 gene can maintain the stability of telomere and slow down telomere shortening by histone deacetylation and chromatin modification, thereby achieving the effects of delaying cell aging and extending lifespan. Specifically, the kiwiberry extract has the ability to increase the expression of the SIRT1 gene by about 1.3 times, thereby reducing damage to skin DNA, protein inactivation, and inflammatory reactions.

In some embodiments, the kiwiberry extract can effectively enhance a DNA repair gene, and the DNA repair gene is the OGG1 gene. The OGG1 gene will increase the secretion of 8-oxoguanine DNA glycosylase in vivo, which in turn excises damaged DNA and aids DNA repair.

In some embodiments, the kiwiberry extract can effectively inhibit the content of ROS. In other words, after a subject takes the kiwiberry extract, skin inflammation and aging caused by ROS can be slowed down, and vicious cycle of inflammation is avoided. For example, the kiwiberry extract can effectively reduce the skin damage caused by ROS by up to 20%.

In some embodiments, the kiwiberry extract can reduce the damage to DNA structure. The damage to cell DNA structure can be caused by either a metabolic process of a cell itself or external stimulation destruction. After a subject takes the kiwiberry extract, the damage to DNA of a cell of the subject can be effectively reduced, and aging is effectively resisted to make the skin look new.

In some embodiments, resisting skin aging is to reduce skin laxity, improve skin elasticity, soothe skin redness, or a combination thereof. In other words, after a subject takes the kiwiberry extract, the skin laxity can be improved, the skin elasticity can be increased, and the skin redness can be soothed.

In some embodiments, the aforementioned subject is a human.

In some embodiments, any of the aforementioned compositions may be a pharmaceutical product. In other words, the pharmaceutical product includes an effective content of kiwiberry extract.

In some embodiments, the pharmaceutical product can be manufactured by using a technology known to those skilled in the art into a dosage form suitable for being enterally, parenterally, orally or topically administrated.

In some embodiments, the enterally or orally administrated dosage form may be, but is not limited to, tablets, troches, lozenges, pills, capsules, dispersible powder or granules, solutions, suspensions, emulsions, syrup, elixirs, slurry, or the like. In some embodiments, the parenterally or topically dosage administrated form may be, but is not limited to, an injection, sterile powder, an external preparation, or the like. In some embodiments, an administration mode of the injection may be, but is not limited to, subcutaneous injection, intraepidermal injection, intradermal injection, or intralesional injection.

In some embodiments, the aforementioned pharmaceutical product may include a pharmaceutically acceptable carrier that is widely used in a drug manufacturing technology. In some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and the like. The type and quantity of the carriers selected falls within the scope of the professional quality and routine technology known to those skilled in the art. In some embodiments, a solvent used as the pharmaceutically acceptable carrier may be water, normal saline, phosphate buffered saline (PBS) and an aqueous solution containing alcohol.

In some embodiments, any of the aforementioned compositions may be an edible product for non-medical use. In other words, the edible product includes a specific content of kiwiberry extract. In some embodiments, the edible product may be a general food, a health-care food, or a dietary supplement.

In some embodiments, the aforementioned edible product can be manufactured by using a technology well known to those skilled in the art into a dosage form suitable for oral administration. In some embodiments, the aforementioned general food may be an edible product itself. In some embodiments, the general food may be, but is not limited to, beverages, fermented foods, bakery products, or seasonings.

In some embodiments, the obtained kiwiberry extract can be further used as a food additive to prepare a food composition containing the kiwiberry extract. Here, it is possible to add the kiwiberry extract in any embodiment during preparation of raw materials by a conventional method, or to add the kiwiberry extract in any embodiment in the food production process to prepare an edible product (i.e., food composition) for human and non-human animal consumption with any edible material.

In some embodiments, the kiwiberry extract contained in the aforementioned composition containing the kiwiberry extract may be liquid or solid. For example, the solid may be powder or a tablet.

In some embodiments, the usage amount of the composition is at least 2.0 g/day of liquid kiwiberry extract.

In some embodiments, the usage amount of the composition is at least 0.2 g/day of solid kiwiberry extract.

### Example 1: Preparation of Kiwiberry Extract

Firstly, fruits of kiwiberry (Actinidia arguta) (origin: New Zealand) were prepared, and roughly crushed into pieces of 12 mm. Subsequently, water was mixed with the fruit pieces of the kiwiberry in a weight ratio of 10:1, and the mixture was extracted at 85±5° C. for 90 min to form a mixed solution containing solids. Here, Brix of the mixed solution at 20° C. was measured to confirm whether it reached, an established specification which is 0.6±0.5 or not.

Subsequently, the mixed solution containing the solids was filtered through a 400-mesh filter to remove fine solids from the mixed solution. Concentration was stopped when the filtered mixed solution was subjected to vacuum concentration by a concentrator (brand/model: BUCHI-Rotavapor R-100) with 1±0.2 kg/cm² of steam at 60° C.±5° C. until Degrees Brix of the solution was 2.5±0.5 to obtain a liquid kiwiberry extract.

### Example 2: Anti-Aging Gene Test

Here, an anti-aging gene detected was an SIRT1 (GeneID: 23411) gene. A medium used was an Eagle's minimum essential medium (hereinafter referred to as MEM medium) containing 10 vol % of fetal bovine serum (FBS; brand: Gibco), 1 mM sodium pyruvate (90%; brand: Gibco), 0.1 mM non-essential amino acids (brand: Gibco), and 1.5 g/L sodium bicarbonate (brand: Gibco). A cell line used was a human skin fibroblast (CCD-966Sk cell, brand: ATCC®, CRL-1881), hereinafter referred to as a CCD-966Sk cell.

The CCD-996SK cells were seeded at 1×10⁵ cells per well into a 6-well culture plate containing 2 ml of MEM media per well and cultured at 37° C. for 24 h, and divided into an experimental group and a control group. Subsequently, the MEM medium was replaced with an experimental medium, and culture was performed for 48 h. The experimental medium in the experimental group was an MEM medium containing 0.125% of kiwiberry extract prepared in Example 1. The experimental medium in the control group was a pure MEM medium (i.e., an MEM medium without the kiwiberry extract).

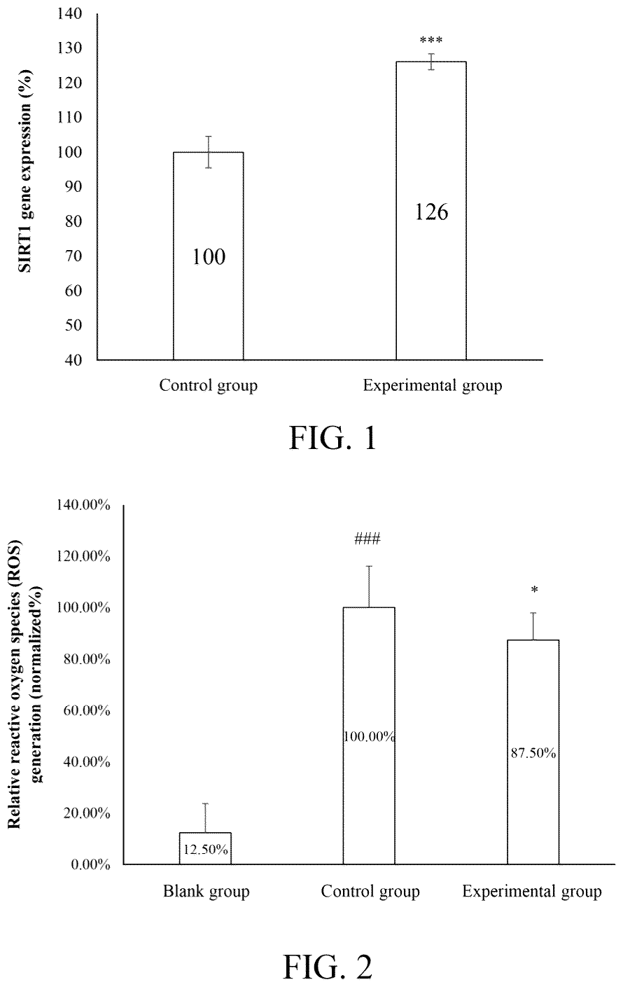

Subsequently, RNA from the CCD-996SK cells cultured in the experimental medium in each group was extracted with an RNA extraction kit (brand: Genemark). 1000 nanograms (ng) of extracted RNA from each group was used as a template, and RNA of each group was translated into cDNA with a cDNA synthesis reagent (purchased from Geneaid, Taiwan) and SuperScriptX III reverse transcriptase. Subsequently, a quantitative real-time reverse transcription polymerase chain reaction was performed by using a KAPA CYBR FAST qPCR kit (KAPA Biosystems) and an ABI Step One Plus real-time PCR system instrument (ABI StepOnePlus™ Real-Time PCR system) in conjunction with SIRT1-F (SEQ ID NO: 1) and SIRT1-R (SEQ ID NO: 2) primers (as shown in Table 1) to quantitatively analyze a target gene. The analysis results are as shown in FIG. 1. Instrument set conditions for the quantitative real-time reverse transcription polymerase chain reaction were: reaction at 95° C. for 23 s, reaction at 60° C. for 30 s, for a total of 40 cycles; and cDNA PCR size of an FBN1 gene was 166 bp. It should be noted that gene expression in FIG. 1 is presented in percentage, where the standard deviation is calculated by using an STDEV formula in Excel software, and statistically significant differences between groups are analyzed by a student t-test. “***” in FIG. 1 is a p value obtained in comparison with that of the control group.

TABLE 1

| Sequence | Sequence | | |
|---|---|---|---|
| number | name | Sequence (5′−>3′) | Length |
| SEQ ID NO: 1 | SIRT1-F | TAGCCTTGTCAGATAAGGAA | 23 |
| | | GGA | |
| SEQ ID NO: 2 | SIRT1-R | ACAGCTTCTTCACAGTCAAC | 25 |
| | | TTTGT | |

See FIG. 1. The expression of an SIRT1 gene of the control group was regarded as 100%, which represents that the expression of the SIRT1 gene in the CCD-996SK cell not treated with the kiwiberry extract was regarded as 100%. Based on this, the expression of the SIRT1 gene in the experimental group was 126%. In other words, the SIRT1 gene in the CCD-996SK cell treated with the kiwiberry extract was increased by about 1.3 times.

Based on this, the kiwiberry extract has an effect of increasing the expression of the SIRT1 gene. In other words, after the kiwiberry extract was taken, the expression of the SIRT1 gene in a subject can be increased, thereby producing SIRT1 protein. The SIRT1 protein has the ability of deacetylation, and participates in multiple physiological regulations in the subject, such as gene expression, metabolism, and aging. Therefore, damage to skin DNA, protein inactivation, and inflammatory reactions can be reduced, and cell aging can be delayed.

### Example 3: Reactive Oxygen Species Test

Here, a medium used was an Eagle's minimum essential medium (hereinafter referred to as MEM medium) containing 10 vol % of fetal bovine serum (FBS; brand: Gibco), 1 mM sodium pyruvate (90%; brand: Gibco), 0.1 mM non-essential amino acids (brand: Gibco), 1% of penicillin-streptomycin (brand: Gibco; Cat. 15140122) and 1.5 g/L sodium bicarbonate (brand: Gibco). A cell line used was a human skin fibroblast (CCD-966Sk cell, brand: ATCC®, CRL-1881), hereinafter referred to as a CCD-966Sk cell. A DCFH-DA solution used was a reaction solution prepared by dissolving 2,7-dichloro-dihydro-fluorescein diacetate (DCFH-DA; product number: SI-D6883, purchased from Sigma) in dimethyl sulfoxide (DMSO, purchased from Sigma, product number: SI-D6883-50MG).

The CCD-996SK cells were seeded at 2×10⁵ cells per well into a 6-well culture plate containing 2 ml MEM medium per well, and cultured at 37° C. for 24 h, and divided into an experimental group, a control group and a blank group. After it was confirmed that the CCD-996SK cells were attached to the bottom of the culture plate, the MEM medium was replaced with an experimental medium, and reaction was performed for 1 h. The experimental medium in the blank group was a pure cell medium. The experimental medium in the control group was an MEM medium containing 1 mM hydrogen peroxide. The experimental medium in the experimental group was an MEM medium containing 1 mM hydrogen peroxide and 0.125% of kiwiberry extract prepared in Example 1.

Subsequently, 1 μL of 5 μg/mL DCFH-DA solutions was added into the experimental medium in each well in each group, and then the CCD-996SK cells were treated at 37° C. for 15 min. After reaction with the DCFH-DA solution, 10 μL of 100 mM hydrogen peroxide (purchased from Sigma) was added into the experimental medium in each group, and reaction was performed at 37° C. for 1 h.

Subsequently, after the experimental medium in each group was removed, the CCD-996SK cells in each group were rinsed twice with 1×DPBS solution (1 mL/well). Then, trypsin (200 μL/well) was added into each well and reaction was performed in dark for 5 min. After the reaction, 1 mL of cell media was added into each well to stop the reaction. The CCD-996SK cells and the cell medium in each well were collected into respective 1.5 mL microcentrifuge tubes, and the microcentrifuge tube containing the CCD-996SK cells and the cell medium was centrifuged at 400×g for 10 min. Supernatant in the microcentrifuge tube in each group was removed after centrifugation, and then pellets of the CCD-996SK cells were re-dissolved in the 1×DPBS solution, and centrifugation was performed at 400×g for 10 min. The supernatant in the microcentrifuge tube in each group was removed again after centrifugation, and then the CCD-996SK cells were re-suspended in dark in 1 mL of 1×PBS solution per centrifuge tube to obtain a cell fluid to be detected in each group.

Flow cytometry (manufacturer: Beckman; Catalog No. 660519) was used to detect a fluorescence signal of DCFH-DA in the cell fluid to be detected in each group. The excitation wavelength of fluorescence detection used was 450 nm to 490 nm, and the emission wavelength was 510 nm to 550 nm. Since DCFH-DA will be hydrolyzed into DCFH (dichloro-dihydro-fluorescein) first after entering CCD-996SK cells, and then DCFH was oxidized by reactive oxygen species (ROS) into DCF (dichloro-fluorescein) that can emit green fluorescence, the fluorescence intensity of the CCD-996SK cells treated with DCFH-DA can reflect the content of the reactive oxygen species (ROS) in the CCD-996SK cells, and whereby the proportion of the number of cells with high expression of the reactive oxygen species (ROS) in the CCD-996SK cells to the number of original cells was known. Since the experiment was conducted in triplicate, the measurement results of the triplicate experiment in each group were averaged to obtain a mean. Then, with the control group as 100%, the proportion relative to the control group was calculated for each group, and presented as a percentage value, as shown in FIG. 2. “###” represents a p value compared with that of the blank group, and “*” represents a p value compared with that of the control group.

See FIG. 2. The cells in the blank group were not treated with the hydrogen peroxide, and their relative ROS normalized generation was 12.50%, which represents a background value of the ROS generation without treatment with the hydrogen peroxide. After the control group was treated with the hydrogen peroxide, its relative ROS normalized generation was 100.00%, indicating that hydrogen peroxide treatment does lead to ROS generation in the cell, which in turn causes subsequent damage to the CCD-996SK cells. After the experimental group was treated with the hydrogen peroxide, its relative ROS normalized generation was 87.50%, but it was decreased by 12.5% relative to that of the control group, indicating that the kiwiberry extract in the cell medium can effectively reduce production or accumulation of the reactive oxygen species (ROS) in the cell, thereby helping the CCD-996SK cells to resist cellular oxidative stress caused by the hydrogen peroxide better. This result indicated that the kiwiberry extract effectively reduced the production or accumulation of the reactive oxygen species (ROS) in the cell, help the cell resist the damage of ROS, and had the ability of resisting oxidation and resisting aging. In other words, the kiwiberry extract was used as a reactive oxygen species scavenger. That is, the kiwiberry extract reduced the content of the reactive oxygen species (ROS) in the cell, thereby reducing oxidative damage caused by the reactive oxygen species in the cells.

Based on this, when a subject takes the kiwiberry extract, skin inflammation and aging caused by ROS can be slowed down, and vicious cycle of inflammation was avoided.

### Example 4: Test of Damage to Cell DNA Structure

Here, in this experiment, DSBs were induced by the hydrogen peroxide (H₂O₂) to phosphorylate histone H2AX, and such phosphorylated forms are referred to as Gamma-H2AX (Ser139) (γH2AX) that can be used as one of indicators of DNA double-strand breaks (DSBs), and can represent the damage to cell DNA structure. Moreover, analysis was performed with enzyme-linked immunosorbent assay (ELISA).

A medium used was an Eagle's minimum essential medium (hereinafter referred to as MEM medium) containing 10 vol % of fetal bovine serum (FBS; brand: Gibco), 1 mM sodium pyruvate (90%; brand: Gibco), 0.1 mM non-essential amino acids (brand: Gibco), 1% of penicillin-streptomycin (brand: Gibco; Cat. 15140122) and 1.5 g/L sodium bicarbonate (brand: Gibco). A cell line used was a human skin fibroblast (CCD-966Sk cell, brand: ATCC®, CRL-1881), hereinafter referred to as a CCD-966Sk cell.

Kit used: Phospho-gamma H2A.X (S139) ELISA kit (purchased from Abcam, Cat. Ab279816), which was placed at room temperature for deicing before the experiment began. Here, the kit contains the following standards used in the experiment and 1× washing solution and other reagents.

Pre-experimental treatment: The CCD-996SK cells were seeded at 2×10⁵ cells per well into a 6-well culture plate containing 2 ml of MEM medium per well, and cultured at 37° C. for 24 h, and divided into an experimental group, a control group and a blank group. After it was confirmed that the CCD-996SK cells were attached to the bottom of the culture plate, the MEM medium was replaced with an experimental medium, and reaction was performed for 1 h. The experimental media in the blank and the control groups were pure MEM media, and the experimental medium in the experimental group was an MEM medium containing 0.0625% of kiwiberry extract prepared in Example 1. After reaction for 1 h, 2 mM hydrogen peroxide was added into the experimental media in the control group and the experimental group, and reaction was performed for 1 h.

Subsequently, after the experimental medium in each group was removed, the CCD-996SK cells in each group were rinsed twice with 1×PBS solution. Then, the CCD-996SK cells were trypsinized, sliced off from the culture plate and collected into respective 1.5 mL microcentrifuge tubes in each group, and the microcentrifuge tube containing the CCD-996SK cells and the cell medium was centrifuged at 400×g for 10 min. Supernatant in the microcentrifuge tube in each group was removed after centrifugation, and then pellets of the CCD-996SK cells were re-dissolved in the 1×PBS solution, and centrifugation was performed at 300×g for 10 min. The supernatant in the microcentrifuge tube in each group was removed again after centrifugation, and the cells were lysed with 120 μL of lysis buffer. Subsequently, the supernatant treated by cell lysis in each group was centrifuged at 4° C. at 13,000×g for 30 min by a micro high-speed low-temperature centrifuge, and then, the supernatant obtained after centrifugation was collected into a 1.5 ml microcentrifuge tube to serve as a sample to be detected for each group.

After protein quantification was performed on the sample to be detected in each group, the protein concentration in each tube was confirmed, and Phospho-gamma H2A.X (S139) content determination was performed by using a Phospho-gamma H2A.X (S139) ELISA kit.

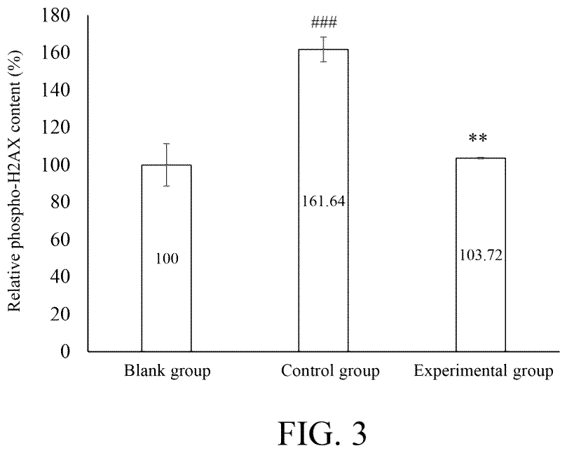

Phospho-gamma H2A.X (S139) content detection flow: 100 μl of standard in kits of corresponding dilution factors was added into corresponding 96-well plates. 100 μl of appropriately diluted sample to be detected was added into the corresponding 96-well plates, and 100 μl of Phospho-gamma H2A.X (S139) antibody prepared was added, and then, the 96-well plate was film-sealed. Subsequently, the film-sealed 96-well plate was placed in a CO₂ incubator at 37° C. for 1 h. After the reaction, the supernatant in each well in each group was removed, and after washing was performed 4 times with a 1× washing solution in the kit, 100 μl of prepared HRP-conjugated anti-rabbit IgG antibody was added. The 96-well plate was film-sealed again and placed in a CO₂ incubator at 37° C. for 1 h. Subsequently, the supernatant in each group was removed, and washing was performed 4 times with the 1× washing solution. Then, a TMB one-step substrate reagent was added and placed at room temperature in dark for 30 min. Finally, after washing was performed again with the 1× washing solution 4 times, 50 μl of stop solution was added into the well in each group, and immediately, an absorbance value at OD450 nm was read on an ELISA instrument. Moreover, the absorbance value of each experimental group minus a background value of average zero (blank) light was calculated to obtain a final average absorbance value of each group (3 replicates). Here, in FIG. 3, “###” represents a p value compared with that of the blank group, and “**” represents a p value compared with that of the control group.

See FIG. 3. The cells in the blank group were not treated with the hydrogen peroxide, and their cell DNA structures were not damaged by ROS, and their relative phospho-H2AX content was used as a 100% reference standard. After the control group was treated with the hydrogen peroxide, the cell DNA structures in the control group were damaged by ROS, and therefore, their relative phospho-H2AX content was 161.64%, indicating that hydrogen peroxide treatment does cause the damage to the DNA structure. After the experimental group was treated with the hydrogen peroxide and the kiwiberry extract, the relative phospho-H2AX content was 103.72%, but it was decreased by 57.92% (with a decrease of about 36%) relative to that of the control group, which represented that the kiwiberry extract in the cell medium effectively reduced production or accumulation of reactive oxygen species (ROS), thereby avoiding or reducing the damage to cell DNA structures.

### Example 5: Human Body Test

To further confirm the influence of the kiwiberry extract on a human body. 50 mL of kiwiberry drink containing 2 g of kiwiberry extract in liquid prepared in Example 1 and 48 g of water per bottle was provided to 12 subjects, each of whom took one bottle of kiwiberry drink on an empty stomach for 4 consecutive weeks. 12 subjects were healthy adults aged 20 and above who wanted to improve their skin conditions.

The 12 subjects would undergo blood collection and analysis and skin quality detection before taking the kiwiberry drink containing the kiwiberry extract (week 0) and 4 weeks after taking the kiwiberry drink containing the kiwiberry extract (week 4). Detection items for blood include expression of an anti-aging gene (SIRT1 gene) and a DNA repair gene (OGG1 gene) in blood. Detection items for skin quality include skin elasticity testing with a skin elasticity detection probe (Cutometer® MPA580 (C+K Multi Probe Adapter System, Germany)), and facial skin photographing with an RBX polarized light technology of a VISIA complexion analysis system (Canfield scientific, USA) for skin redness detection.

It should be noted that statistical analysis was performed through a student t-test on statistically significant differences between the measurements from week 0 and week 4. “*/**” represents a p value compared with that at week 0.

### Example 5-1: Analysis of Expression of Anti-Aging Gene and DNA Repair Gene in Blood

Here, anti-aging genes detected were an SIRT1 gene (Gene ID: 23411) and an OGG1 gene (Gene ID: 4968).

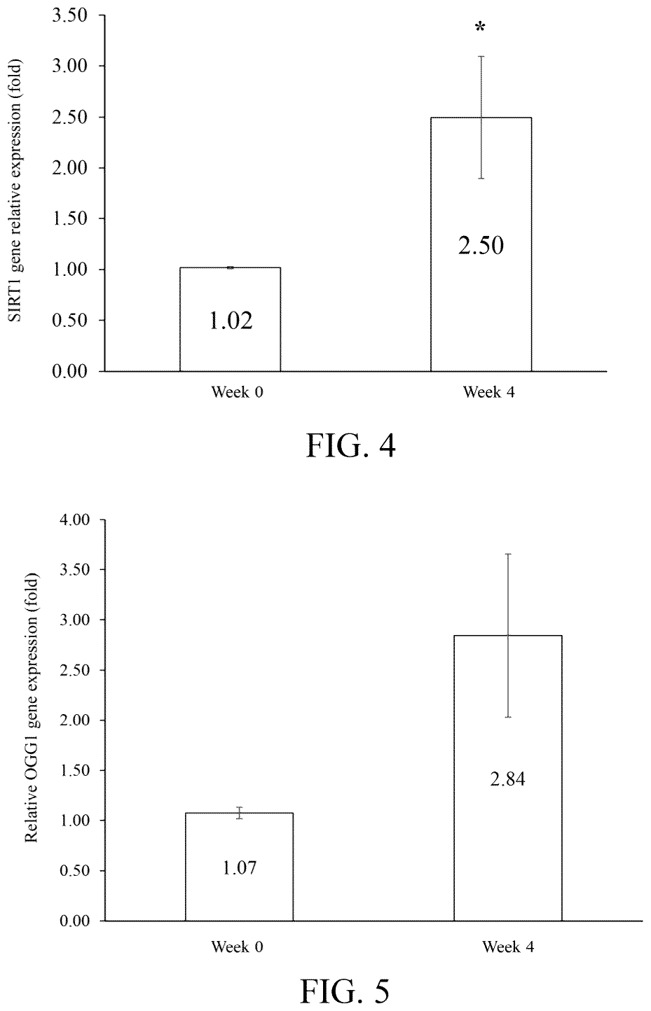

Firstly, extracted venous blood was centrifuged at of 300×g for 15 min. 2 mL of leukocytic cream of buffy coat was taken from the centrifuged venous blood, and the leukocytic cream was diluted to 4 mL with 2 mL of phosphate buffer (1×PBS; hereinafter referred to as 1×PBS buffer). Subsequently, the diluted leukocytic cream was slowly added to a centrifuge tube containing 3 mL of cell separation solution (Ficoll-Paque Plus cell separation solution), in which the diluted leukocytic cream and the cell separation solution needed to be layered, and could not be mixed during addition. Subsequently, the centrifuge tube holding the stratified diluted leukocytic cream and the cell separation solution was centrifuged at 400×g for 40 min, and supernatant was removed after centrifugation. 2 mL to 3 mL of peripheral blood mononuclear cells (hereinafter referred to as PBMC) in an intermediate layer in the aforementioned centrifuged centrifuge tube was taken. PBMC was rinsed with 3 times the volume of 1×PBS buffer, and then, the rinsed PBMC was mixed with the aforementioned 1×PBS buffer homogeneously to form a PBMC mixed solution. Subsequently, the PBMC mixed solution was centrifuged at 300×g for 10 min to form PBMC supernatant and PBMC pellets, and the PBMC pellets were lysed with 600 μL of RNA lysis buffer, and then RNA was extracted. Subsequently, as shown in the steps in Example 2, the extracted RNA was reverse-transcribed into cDNA, and quantitative real-time reverse transcription polymerase chain reaction was performed on cDNA with two sets of primers (SEQ ID NO: 1 and SEQ ID NO: 2, SEQ ID NO: 3 and SEQ ID NO: 4) in table 2 to observe expression of the SIRT1 gene and the OGG1 gene in the blood at week 0 and week 4, as shown in FIG. 4.

TABLE 2

| Target | Primer | Sequence | | Primer |
|---|---|---|---|---|
| gene | name | number | Sequence | length |
| SIRT1 | SIRT1-F | SEQ ID NO: 1 | TAGCCTTGTCAGAT | 23 |
| | | | AAGGAAGGA | |
| | SIRT1-R | SEQ ID NO: 2 | ACAGCTTCTTCACA | 25 |
| | | | GTCAACTTTGT | |
| OGG1 | OGG1-F | SEQ ID NO: 3 | CAAGCGACTCCACC | 20 |
| | | | CTCCTA | |
| | OGG1-R | SEQ ID NO: 4 | GCCCTTTGGAACCC | 20 |
| | | | TTTCTG | |

In Table 2, F is a forward prime and R is a reverse primer.

It should be noted that gene expression in FIG. 4 and FIG. 5 is presented in relative rate, where the standard deviation is calculated by using an STDEV formula in Excel software, and statistically significant differences between groups are analyzed by a student t-test. In FIG. 4, “*” represents that a p value is less than 0.05 in comparison with that at week 0.

See FIG. 4. When the relative gene expression rate of the SIRT1 gene in the 12 subjects at week 0 was 1.02, the relative gene expression rate of the SIRT1 gene at week 4 was 2.50 (increased by 2.5 times). From this, it could be seen that when the subjects take the kiwiberry drink containing 2 g of kiwiberry extract daily for 4 consecutive weeks, the expression of the SIRT1 gene in the blood of the subject was increased, indicating that the expression of protein encoded by the SIRT1 gene was increased. Moreover, the SIRT1 gene, as a downstream gene of NAD⁺, is related to the activity of mitochondria. When the SIRT1 gene in the blood rises, it reflects that the content of NAD⁺ in the blood was increased, thus achieving a function of resisting aging. In other words, the expression of the SIRT1 gene in the blood was increased by taking the kiwiberry extract, which indicated that the kiwiberry extract have a potential of resisting aging.

See FIG. 5. When the relative gene expression rate of the OGG1 gene in the 12 subjects at week 0 was 1.07, the relative gene expression rate of the OGG1 gene at week 4 was 2.84 (increased by 2.84 times). From this, it could be seen that when the subjects take the kiwiberry drink containing 2 g of kiwiberry extract daily for 4 consecutive weeks, the expression of the OGG1 gene in the blood of the subject was increased, and the OGG1 gene increased the secretion of 8-oxoguanine DNA glycosylase, and excised damaged DNA and helped DNA repairing.

### Example 5-2: Analysis of Skin Condition

### Skin Detection Principle:

Skin elasticity and laxity: The test principle was based on principles of suction and stretching. Negative pressure was generated on the surface of tested skin to suck the skin into a test probe. The depth of the skin sucked into the probe was detected with an optical test system, and the skin elasticity and the laxity were analyzed and calculated with software.

Skin redness: Facial skin was photographed with the RBX polarized light technology to detect deep blood vessels or hemoglobin in the skin. The higher the measurement, the more severe the skin redness.

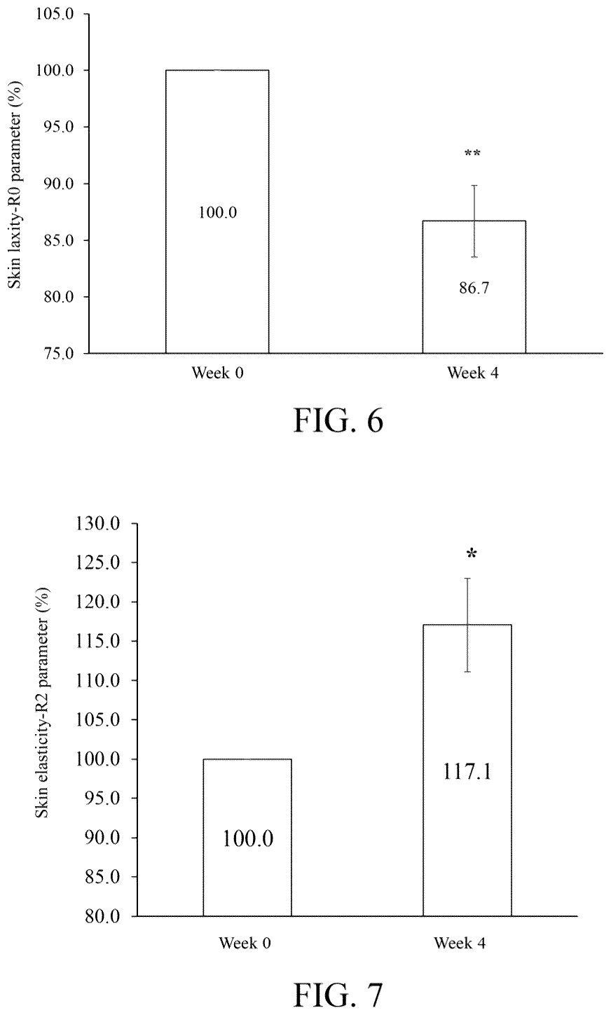

See FIG. 6. The average skin laxity-RO parameter percentage (%), detected by the skin elasticity detection probe, of the 12 subjects before taking the kiwiberry extract (week 0) was regarded as 100%. After the subjects took the kiwiberry extract for 4 consecutive weeks, the average skin laxity percentage of the subjects was decreased to 86.7%. In other words, the skin laxity percentage of these subjects was decreased by 13.3% after taking the kiwiberry drink containing 2 g of kiwiberry extract for 4 consecutive weeks compared with that before taking the kiwiberry extract (week 0). From this, it could be seen that the skin laxity of a subject was reduced, the skin condition of the subject was improved, and the skin was made tight and elastic by taking the kiwiberry extract for a long time.

See FIG. 7. The average skin elasticity-R2 parameter percentage (%), detected by the skin elasticity detection probe, of the 12 subjects before taking the kiwiberry extract (week 0) was regarded as 100%. After the subjects took the kiwiberry extract for 4 consecutive weeks, the average skin elasticity percentage of the subjects was increased to 117.1%. In other words, the skin elasticity percentage of these subjects was increased by 17.1% after taking the kiwiberry drink containing 2 g of kiwiberry extract for 4 consecutive weeks compared with that before taking the kiwiberry extract (week 0). From this, it could be seen that the skin elasticity was improved and the skin was made tight by taking the kiwiberry extract for a long time.

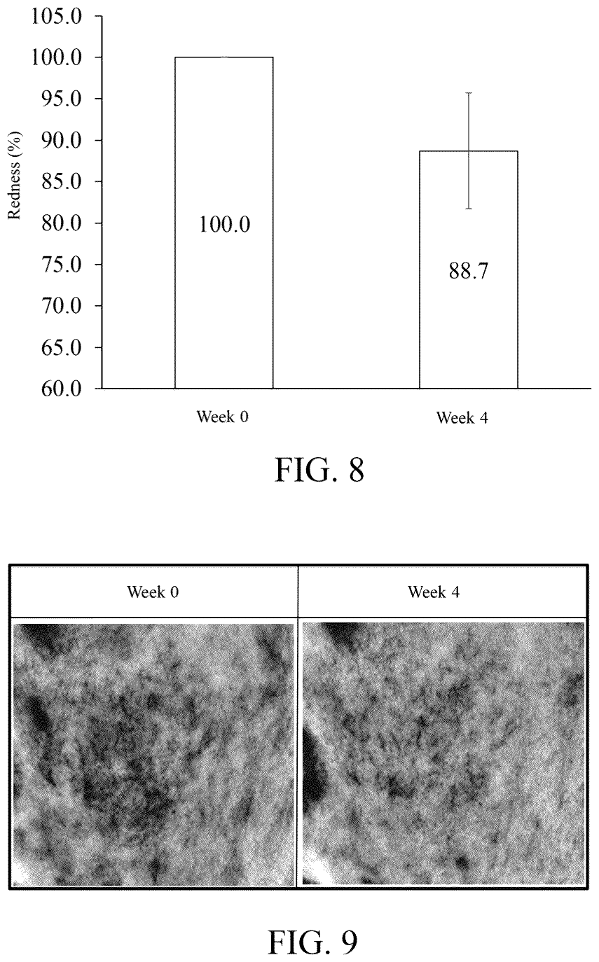

See FIG. 8. The average skin redness percentage (%), detected by a VISIA high-tech digital skin detector, of one of the subjects before taking the kiwiberry extract (week 0) was regarded as 100%. After the subjects took the kiwiberry extract for 4 consecutive weeks, the average skin redness percentage of the subjects was decreased to 88.7%. In other words, the skin redness percentage of these subjects was decreased by 11.3% after taking the kiwiberry drink containing 2 g of kiwiberry extract for 4 consecutive weeks compared with before taking the kiwiberry extract (week 0). From this, it could be seen that the skin redness was soothed by taking the kiwiberry extract for a long time.

See FIG. 9. One of the subjects was photographed with the VISIA high-tech digital skin detector for skin redness, which showed that the skin redness of the subject was reduced after the subject took the kiwiberry extract for 4 consecutive weeks.

In summary, the composition for resisting skin aging is prepared from the kiwiberry extract of the embodiments of the present invention. The kiwiberry extract is obtained by extracting the fruits of kiwiberry with water. In some embodiments, the kiwiberry extract has the effects of reducing skin damage, reducing damage to DNA structure, reducing skin laxity, improving skin elasticity, soothing skin redness, and the like.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for resisting skin aging, comprising administrating to a subject in need thereof a composition comprising an effective dose of kiwiberry extract, wherein the kiwiberry extract is obtained by extracting kiwiberry (Actinidia arguta) with water.

2. The method according to claim 1, wherein the kiwiberry extract reduces skin damage, reduces damage to DNA structure, or a combination thereof to achieve the effect of resisting skin aging.

3. The method according to claim 2, wherein the kiwiberry extract achieves reduction of skin damage by reducing reactive oxygen species (ROS) in the subject.

4. The method according to claim 2, wherein the kiwiberry extract achieves reduction of damage to DNA structure by increasing expression of an NAD-dependent deacetylase (Sirtuin-1, SIRT1) gene and/or an 8-hydroxyguanine glycosylase (OGG1) gene in the subject.

5. The method according to claim 1, wherein the kiwiberry extract reduces skin laxity to achieve the effect of resisting skin aging.

6. The method according to claim 1, wherein the kiwiberry extract improves skin elasticity to achieve the effect of resisting skin aging.

7. The method according to claim 1, wherein the kiwiberry extract soothes skin redness to achieve the effect of resisting skin aging.

8. The method according to claim 1, wherein the kiwiberry extract is obtained by extracting the kiwiberry in water at 70° C. to 90° C. for 90 min to 120 min.

9. The method according to claim 8, wherein a weight ratio of the kiwiberry to the water is (1 to 5):(5 to 10).

10. The method according to claim 8, wherein when the composition is in liquid form, the effective dose of the composition is 2 g/day, or when the composition is in solid form, the effective dose of the composition is 0.2 g/day.

11. The method according to claim 1, wherein the kiwiberry extract is obtained by extracting the kiwiberry in water at 70° C. to 90° C. until a Brix degree thereof reaches 0.6±0.5 at 20° C.

12. The method according to claim 11, wherein the weight ratio of the kiwiberry to the water is (1 to 5):(5 to 10).

13. The method according to claim 11, wherein when the composition is in liquid form, the effective dose of the composition is 2 g/day, or when the composition is in solid form, the effective dose of the composition is 0.2 g/day.


SEQUENCE LISTING (4 sequences)

SEQ ID NO: 1 - DNA, 23 bp, synthetic construct
  source 1..23: mol_type = other DNA
tagccttgtcagataaggaagga

SEQ ID NO: 2 - DNA, 25 bp, synthetic construct
  source 1..25: mol_type = other DNA
acagcttcttcacagtcaactttgt

SEQ ID NO: 3 - DNA, 20 bp, synthetic construct
  source 1..20: mol_type = other DNA
caagcgactccaccctccta

SEQ ID NO: 4 - DNA, 20 bp, synthetic construct
  source 1..20: mol_type = other DNA
gccctttggaaccctttctg